(19) United States
(12) Patent Application Publication
(10) Pub. No.: US 2009/0263490 A1
(43) Pub. Date: Oct. 22, 2009

(54) Microsphere beeswax for mammalian dietary fat mitigation

(76) Inventor: Allan Yang Wu, (US)

Correspondence Address: ALLAN Y. WU, 447 VIA MILANO, CATHEDRAL CITY, CA, 92234, US

(21) Appl. No.: 12/386,404

(22) Filed: Apr. 18, 2009

Related U.S. Application Data
(60) Provisional application No. 61/124,772, filed on Apr. 19, 2008.

(51) Int. Cl. A61K 35/24; A61K 9/16; A61P 1/00

(52) U.S. Cl. 424/490; 424/537

(57) ABSTRACT

A device composed of a plurality of beeswax microspheres are contained within a digestible capsule intended for the alimentary tract of the mammal. A method in which the device is ingested orally into the digestive system. A method in which digestive enzymes remove the outside capsule and beeswax microspheres are then dispersed within the gastrointestinal tract. The beeswax microsphere is then able to come in contact with free fats, lipids, cholesterol and lipophillic toxins within the gut and absorb them into the lumen of the microsphere. The beeswax microsphere along with all the fats, lipids, cholesterol and lipophillic toxins contained within the microsphere transits through the remainder of the gut undigested and are eventually excreted from the rectum and bowel by fecal elimination.

### CROSS-REFERENCE TO RELATED APPLICATION

This application claims the benefit of U.S. Provisional Patent Application No. 61/124,772 filed Apr. 19, 2008; this provisional application is incorporated herein by reference in its entirety.

### FIELD OF THE INVENTION

The present invention relates to a device and method for absorption of dietary fats from within the gastrointestinal tract of mammalian species. In one exemplary application the fat is absorbed from the human gastrointestinal tract.

### BACKGROUND OF THE INVENTION

Obesity is a problem reaching epidemic proportions world wide. There are several potential remedies both medical and surgical. Lipid lowering drugs block enzymes in the production of undesirable fats and cholesterols. Surgery decreases absorption and consumption through either gastric bypass or gastrointestinal restriction (or banding) of some form. Both medical and surgical methods are with considerable side effects. Medicinal treatment of lipids and cholesterol can result in harmful elevation of liver enzymes and liver damage. Medicinal weight loss regimens have resulted also in non-reversible cardiopulmonary problems such as persistent pulmonary hypertension with FenPhen derivatives thereof.

Surgical treatment can result in higher rates of suicide, chronic diarrhea from “dumping syndrome” (as with gastric bypass) and severe gastroesophageal reflux (as with restrictive procedures). A more natural and safe method of weight loss and cholesterol and lipid control is needed.

Petroleum hydrocarbons, like dietary fats and lipid require remediation after inadvertent spills as previously described in U.S. Pat. No. 6,699,390. Processed microsphere beeswax carries the unique capability of absorbing long chain petroleum hydrocarbons. Beeswax is also considered biologically inert or indigestible to the human gastrointestinal tract (The Fats of Life). The use of specialty formulated microsphere beeswax therefore presents a novel device and method of mitigating human absorption of long chain hydrocarbons and lipophillic substances such as dietary fats, cholesterols and lipids.

### SUMMARY OF THE INVENTION

Beeswax can be fashioned into microspheres by many manufacturing processes. Beeswax alone has hydrophobic properties which contributes to its natural affinity and capability of scavenging long chain hydrocarbons such as fat. Additionally, the increased exposed surface area by the creation of microspheres that contributes to considerably greater fat scavenging capabilities.

The beeswax microspheres can be packaged by any suitable means necessary to assure tolerability and ingestability into the gastrointestinal tract, such as a capsule, caplet, tablet or gel cap. After release from the protective coating, the BMS can distribute through out the intestinal tract. By being in contact with the mixture of chewed and partially digested food, the BMS is then capable of absorbing or solvating the hydrophobic compounds such as fat, cholesterol and other lipophillic substances, since it is less polar that water, the predominant liquid and solvent within the gastrointestinal tract.

The BMS now filled with lipids, fats, cholesterol and lipophillic substances will then transit through the remaining intestinal tract. Since the lipids, fats, cholesterol and lipophillic substances are essentially trapped within the BMS, they cannot come into contact with the brush border of the intestinal tract and therefore cannot become absorbed into the host. The BMS continue into the lower intestinal tract where finally they are eliminated by fecal elimination.

Those familiar with the art and field of cellular biology, pharmacology, physiology, and food science are aware the invention and all potential embodiments may be modified and composed of alternate materials to enhance or improve the process of selection and isolation or clinical outcomes and/or laboratory applications. Similarly, a method of using the device may include a variety of different steps.

### BRIEF DESCRIPTION OF DRAWINGS

FIG. 1. Cross sectional view of BMS.

FIG. 2. Cross sectional view of BMS after absorbing fat.

FIG. 3. Capsule cross section containing BMS for oral and/or enteral consumption

FIG. 4. Conceptual illustration of typical treatment with encapsulated BMS and passage through a human digestive tract.

### DETAILED DESCRIPTION

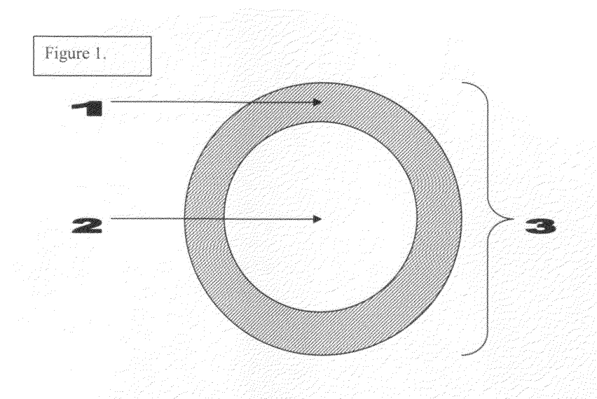

FIG. 1. Cross sectional view of BMS, whereby 3 represents the entire microsphere, 1 represents the microsphere wall and 2 depicts an empty center space prior to absorbing long chain hydrocarborns, lipids, cholesterol and/or fats.

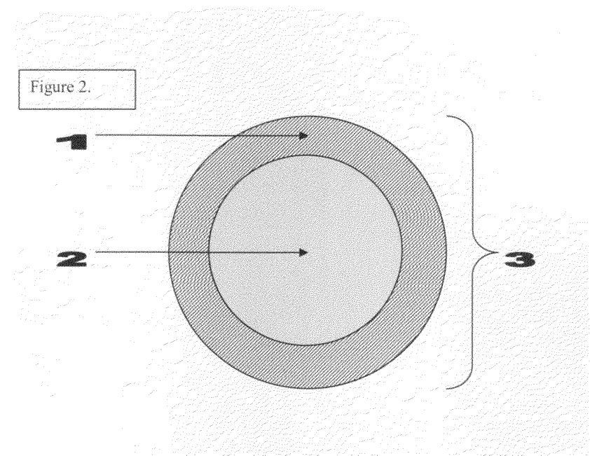

FIG. 2. Cross sectional view of BMS after absorbing fat, whereby 3 represents the entire microsphere, 1 represents the microsphere wall and 2 depicts a filled center space after absorbing long chain hydrocarborns, lipids, cholesterol and/or fats.

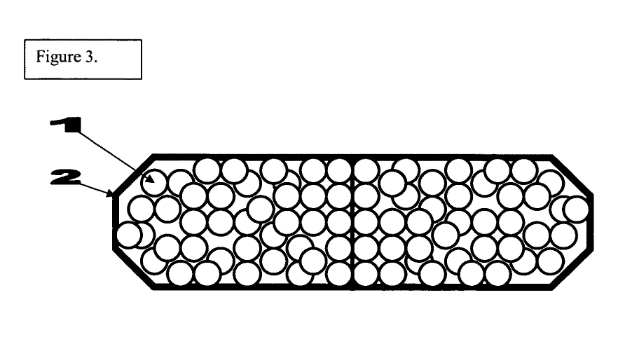

FIG. 3. Capsule cross section containing BMS for oral and/or enteral consumption, whereby 1 represents the functional unit of BMS encapsulated within a digestible coating 2.

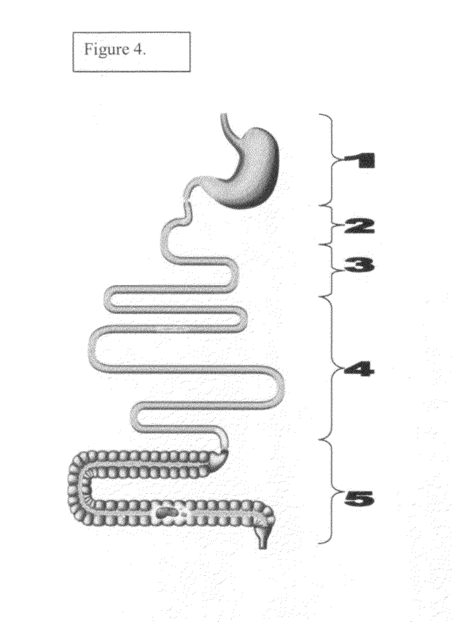

FIG. 4. Conceptual illustration of typical treatment with encapsulated BMS whereby the capsule is ingested orally and the capsule coating is digested in the stomach 1 and passing through the duodenum 2, jejunum 3 and ileum 4. During the transit through the upper and mid GI tract 1 though 4, the MBS mix with and come into contact and absorb lipid and lipophillic substances. Finally the BMS is passed through the large bowel 5 and excreted by fecal elimination.

### EXAMPLE 1

Production of BMS.

Yellow beeswax (12 oz) was melted at a temperature of 70 deg C. and quickly transferred in a molten state to the pre-warmed vial of an atomizer. Atomizer was sprayed into a cooled 4 deg C. chamber. BMS were gently mixed with powdered starch (10 mg) to prevent sticking to one another. BMS were confirmed under 40× microscopy. BMS were in a range from 10 to 200 microns in diameter. BMS were cut in half with fine 30 gauge needles to observe a hollow spherical shape.

### EXAMPLE 2

Confirmation of Lipid Uptake.

A source of animal fat (Butter 6 oz total) was slowly heated until complete melting and placed in sterile water (250 cc) at 37 deg C. This mixture was pulse vortexed×1 minute and allowed to sit for 5 minutes for the lipid water interface to form. The 1 mg of 1× Oil red O was added to the water and allowed to stain the animal fats in the supernatant phase. Again the mixture was pulse vortexed for 1 minute and allowed to sit for 5 minutes at 37 deg C. for the lipid-water interface to form. Red staining was only appreciated in the upper phase. BMS (50 cc) was added to the vial. The vial was gently vortexed for 20 seconds and allowed to sit at 37 deg C. for 2 hours as the lipid-water interface was allowed to form again. Supernatant was aspirated from the vial and analyzed under light microscopy under 40× magnification. BMS were rinsed with sterile water until no residual red dye was appreciated. BMS were once again dissected open carefully under stereoscope magnification at 40× with 30 gauge needles. Red dye was appreciated within the center of the microsphere. The remaining microspheres were crushed to release internal contents and separated from the beeswax. Melting points for the beeswax and internal contents were reconfirmed. Internal contents were positive for a melting point of 32 deg C. consistent with butter. Crushed BMS was found to have a melting point of (63 deg C.) consistent with beeswax.

### EXAMPLE 3

Consumption of Encapsulated BMS Prior to and after a Fatty Meal.

BMS were produced in the same fashion as in example 1. Approximately 100 mg of BMS were packaged into digestible capsule shells (Wonder Labs 00). 2 capsules were taken prior to and after a standardized meal (980 calories total) high in animal fat (400 calories). 2 hour post prandial, whole blood (10 cc) was collected by standard phlebology from the right antecubitum. Blood was aspirated into a small microcapillary tube (Daigger Labs) and centrifuged for 5 minutes at 800 rpm. Fat cap above the serum phase was measured with a caliper using a modified Allen method. This experiment was repeated a total of 3 times in the same subject with 1 week between each repeat experiment. Averages of treatment (post-pradial with BMS) versus no treatment (pre-prandial and no BMS) are represented in table 1.

### REFERENCES

1. Use of Beeswax to degrade hydrocarbons: Griffin U.S. Pat. No. 6,699,390 Mar. 2, 2004

2. The Fats of Life page 117 Caroline Margaret Pond, Cambridge University Press 1998 (“Beeswax simply passes through the gut.”)

3. Stable Compositions for parenteral administration and their use: Cady U.S. Pat. No. 6,340,671 B1 Jan. 22, 2002

I claim:

1. A microsphere composed of beeswax modified for mammalian oral consumption.

2. The microsphere in claim 1 is capable of absorbing: lipids, fats, cholesterol and lipophillic toxins, lipophillic chemicals, lipophillic drugs and lipophillic molecules.

3. The microsphere in claim 1 manufactured in a diameter size range approximately 1-10,000 microns in diameter.

4. The microsphere in claim 1 manufactured with a wall thickness of 1 to 4,500 microns thick.

5. A collection of microspheres as in claim 1 approximately the same size.

6. The composition of claims 1 through 5 admixed with minute quantities of starch or any non-toxic material to prevent the microspheres from sticking together.

7. The composition of claims 1 through 6 encapsulated within an orally consumable and digestible capsule.

8. The composition of claims 1 through 6 in the form of a caplet, tablet, liquid, gel, gelcap or powder.

9. The same composition in claim 7 and 8 may be ingested prior to, during and after meals.

10. The same microsphere in claim 1 may be modified to decrease digestive enzyme degradation of the microsphere wall.

11. The same microsphere in claim 1 may be modified in composition to increase melting point temperatures and/or to prevent loss of spherical structure and wall integrity within the mammalian gut.

12. The microsphere in claim 1 composed of any suitable material that enhances fat, cholesterol and lipophillic toxin absorption and minimizes breakdown within the mammalian gut.

13. The microsphere in claim 1 containing in the center enzymes, medicines or compounds which degrade or neutralize fats, lipids and/or cholesterols within an intact microsphere when in vivo.

14. The microsphere in claim 1 may be coated on the exterior surface with antidiarrheal medication(s), herbal(s) or medicinal formulation(s).

15. The microsphere in claim 1 may be composed of any suitable wax or wax-type material that is non-digestible by the mammalian gut, while at the same time, having the functional capability of absorbing: lipids, fats, cholesterol and lipophillic toxins, lipophillic chemicals, lipophillic drugs and lipophillic molecules.

16. The composition in claim 1 in which the exterior shape may be modified to increase absorption of: lipids, fats, cholesterol and lipophillic toxins, lipophillic chemicals, lipophillic drugs and lipophillic molecules.

17. A method for treating or preventing dietary fat, cholesterol, lipid and lipophillic toxin, lipophillic chemical, lipophillic drug and/or lipophillic molecule absorption through the gastrointestinal tract, which comprises orally administering a clinically effective amount of the microspheres and their variations as listed in claims 1 through 16 to a human or mammal.
